(19) United States
(12) Patent Application Publication
(10) Pub. No.: US 2015/0065776 A1
(43) Pub. Date: Mar. 5, 2015

(54) INCREASED RADIOTHERAPY PATIENT THROUGHPUT

(71) Applicant: Moshe Ein-Gal, Ramat Hasharon (IL)

(72) Inventor: Moshe Ein-Gal, Ramat Hasharon (IL)

(21) Appl. No.: 14/013,137

(22) Filed: Aug. 29, 2013

(51) Int. Cl. A61N 5/00

(52) U.S. Cl. CPC A61N 5/00
USPC 600/1

(57) ABSTRACT

A system and method for radiotherapy patient throughput is described. The system includes carts for supporting and moving patients between stations. A mounting and immobilization station allows mounting and immobilizing the patients on the carts. A registration station produces imaging data related to the patients. A waiting station provides waiting space for the carts. A treatment station irradiates the patients on the carts according to a treatment plan. A dismounting station allows releasing and dismounting the patients from the carts.

### FIELD OF THE INVENTION

The present invention generally relates to radiotherapy or radiosurgery systems and methods, and particularly to a system and method for increasing radiotherapy or radio surgery patient throughput.

### BACKGROUND OF THE INVENTION

Treatment planning for radiotherapy or radiosurgery (the terms being used interchangeably throughout) typically starts with 3D imaging of an immobilized patient (e.g. CT (computerized tomography), MRI (magnetic resonance imaging) or PET (positron emission tomography), followed by delineating targets and organs-at-risk. Subsequently, image-related dose distributions are calculated and a treatment plan is optimized.

Ideally, the positions of the patient in both the imaging and treatment systems should be identical and perfectly registered with each other so as to accurately implement the treatment plan. However, in the real world there are discrepancies. For example, a significant time lapse between imaging and treatment may lead to localization discrepancies.

Different approaches have been tried to deal with this problem. For example, image guided radiotherapy (IGRT) seeks to improve dose delivery by localizing a patient at the treatment location just prior to treatment.

Adaptive radiotherapy seeks to update a treatment plan according to the patient's 3D imaging obtained close to treatment time. A dedicated cone beam CT (CBCT) scanner is mounted on a treatment gantry, thus keeping a patient in a fixed position for 3D imaging and treatment. However, CBCT image quality is generally inferior to that of general purpose diagnostic CT.

In-room CT amounts to performing CT scanning by a high quality diagnostic scanner in the treatment room so as to significantly reduce time lapse between imaging and treatment. An immobilized patient is transported between the imaging and treatment stations. Alternatively, a CT scanner is moved into and out of the treatment station (CT on rails).

The total time spent by a patient in a treatment room is a major factor in determining what is called “patient throughput”, i.e., the number of patients treatable by a treatment system in a day. However, since IGRT, CBCT and in-room CT all take place in the treatment room, much time is spent there on mounting, immobilization, localization and dismounting of the patients.

In the prior art, as seen in FIG. 1, there are N patients (P(1), P(2) . . . P(N)) in hospital rooms. One patient at a time is treated in the radiotherapy room, including the following acts: mounting and immobilization, which takes time T1(P(1)) for patient P(1), T1(P(2)) for patient P(2), and so forth; imaging (that is, imaging, localization and positioning), which takes time T2(P(1)) for patient P(1), T2(P(2)) for patient P(2), and so forth; waiting for treatment, which takes time T3(P(1)) for patient P(1), T3(P(2)) for patient P(2), and so forth; treatment (irradiation), which takes time T4(P(1)) for patient P(1), T4(P(2)) for patient P(2), and so forth; and dismounting, which takes time T5(P(1)) for patient P(1), T5(P(2)) for patient P(2), and so forth.

Non-irradiating activities are mounting and immobilization, imaging (registration), waiting and dismounting. Non-irradiating time is the combined respective times of the non-irradiating activities. Thus, in the prior art, the total time Tₜₒₜₐₗ for dealing with N patients by applying to each patient irradiating and non-irradiating activities, amounts to:

Tₜₒₜₐₗ=N*(irradiating time per patient)+N*(non-irradiating time per patient), assuming that irradiating times are equal for all patients and non-irradiating times are also equal for all patients.

In general, for non-equal times in the prior art, the total time for dealing with all the patients, including mounting and immobilization, imaging, waiting, treatment and dismounting, amounts to:

$$Total time = {\sum }_{i = 1}^{N} ( T 1 ( P ( i ) ) + {\sum }_{i = 1}^{N} ( T 2 ( P ( i ) ) + {\sum }_{i = 1}^{N} ( T 3 ( P ( i ) ) + {\sum }_{i = 1}^{N} ( T 4 ( P ( i ) ) + {\sum }_{i = 1}^{N} ( T 5 ( P ( i ) )$$

### SUMMARY OF THE INVENTION

The present invention seeks to provide novel systems and methods for increasing radiotherapy or radiosurgery patient throughput as is described more in detail hereinbelow.

A radiotherapy system enables patients to be mounted on respective carts and immobilized in a mounting station. Each cart carrying an immobilized patient is moved to a registration station where the immobilized patient is registered to the cart, i.e., is localized and positioned for treatment (translated and rotated relative to the cart on the cart) according to a respective treatment plan.

Following registration, each registered patient is moved by the respective cart to a waiting station and from there, in due time, to a treatment station where the cart is registered to the treatment device, and the patient—already registered to the cart—is thus registered to the treatment device according to the respective treatment plan.

Following treatment, each cart moves to a dismounting station for releasing and dismounting the patient treated on that cart.

While patients are treated in treatment stations, other patients may be registered to respective carts and moved to the waiting station. Patient throughput is increased since time spent in a treatment station is dedicated to treatment only. Stations may share the same location.

### BRIEF DESCRIPTION OF THE DRAWINGS

The present invention will be understood and appreciated more fully from the following detailed description, taken in conjunction with the drawings in which:

FIG. 1 is a simplified block diagram of a prior art system and method for radiotherapy patient throughput; and

FIG. 2 is a simplified block diagram of a method for radiotherapy patient throughput, in accordance with an embodiment of the present invention.

### DETAILED DESCRIPTION OF EMBODIMENTS

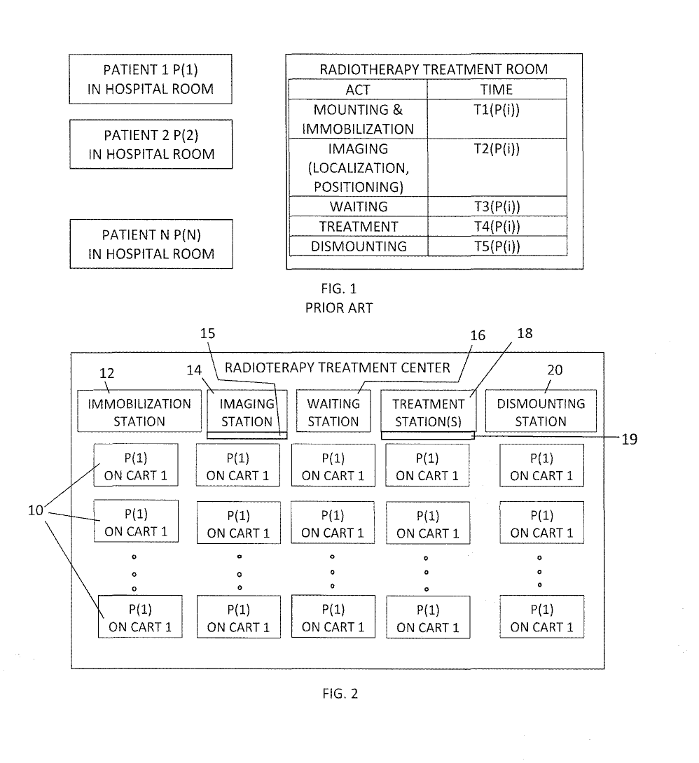

Reference is now made to FIG. 1, which illustrates a system and method for radiotherapy patient throughput, in accordance with a non-limiting embodiment of the present invention.

The system includes carts 10 for a plurality of patients. The term “cart” encompasses any device suitable for supporting and moving a patient, such as but not limited to, a table, stretcher or bed with wheels, and may include devices for translating and rotating the patient for adjusting the position of the patient for imaging and registration purposes and the like, such as but not limited to, turntables, linear actuators, servomotors, optical aligners, etc.

Each cart 10 is dedicated and arranged to support and move a patient between stations. The first station is a mounting and immobilization station 12, which allows mounting and immobilizing the patients on carts 10.

An imaging and registration station 14 includes an imager 15 (e.g. CT, MRI or PET) that produces imaging data related to the patients on carts 10. As with many commercially available medical imagers, imager 15 includes image registration. As is well known in the art, in order to compare images, the contents of the images must be in alignment. The process of finding the correspondence between the contents of images is called image registration. This is achieved by optimizing a measure of similarity between images, such as the position of corresponding landmarks or the similarity in intensity of corresponding anatomical structures. For example, image registration may include global translations and rotations to align the images. If images have become deformed, image registration may further include non-rigid or elastic registration. This is necessary for registration of images of different individuals (who have different anatomies) or images of a single individual when an anatomical change has taken place.

Various image registration techniques are known from the prior art, and many are summarized in the book “Medical Image Registration” by Josef Hajnal or in “Handbook of Medical Image Processing”, Academic Press, 2000.

A waiting station 16 provides waiting space for carts 10 while waiting for treatment.

A treatment (irradiating) station 18 is provided for irradiating the patients on carts 10 according to a treatment plan. Optionally, more than one treatment station 18 can be provided for treating more than one patient simultaneously. The treatment station 18 includes an irradiator 19 operable to irradiate the immobilized patients on carts 10 according to respective treatment plans. The irradiator 19 includes image registration (as described above), optical alignment or mechanical coupling (such as but not limited to, turntables, linear actuators, servomotors, optical aligners, etc.), or any combination thereof, for registering the carts 10 to the irradiator 19.

The final station is a dismounting station 20, which allows releasing and dismounting the patients from carts 10. All stations, except the treatment station(s) 18, share a common route from mounting and immobilizing the patients on carts 10, imaging the patients, and waiting. This common route is shielded in accordance with a predetermined safety level determined by local safety codes.

The radiotherapy system enables a plurality of patients to be mounted on respective carts 10 and immobilized in mounting station 12. The carts 10 then sequentially move to imaging station 14 where each immobilized patient is localized and positioned for treatment (which may include translation and rotation on cart 10) according to a respective treatment plan.

Following localization, each localized patient is moved on his/her cart 10 to waiting station 16 and from there, in due time, to treatment station 18.

Following treatment, each cart 10, with its patient, moves to dismounting station 20 for releasing and dismounting the treated patient.

In contrast with the prior art, in the present invention, in order to significantly save time, while patients are treated in treatment station(s) 18, other patients may be localized and moved to the waiting station. Patient throughput is increased since the time spent in treatment station 18 is dedicated to treatment only.

Thus, in the present invention, wherein irradiating and non-irradiating activities for different patients take place simultaneously, the total time is significantly shortened. For example, if non-irradiating time per patient is shorter than irradiating time per patient, then the total time for N patients, assuming irradiating times are equal for all patients and non-irradiating times are also equal for all patients, is

Tₜₒₜₐₗ₌1*(non-irradiating time)+N*(irradiating time);

which amounts to a saving of (N−1)*(non-irradiating time), compared to the prior art.

In general, for non-equal times, in the present invention, the total time for dealing with all the patients, including mounting and immobilization, imaging, waiting, treatment and dismounting, amounts to:

$$Total time = {\sum }_{j = 1}^{S} ( T j ( P ( 1 ) ) + {\sum }_{i = 2}^{N} ( T 3 ( P ( i ) ) + {\sum }_{i = 2}^{N} ( T 4 ( P ( i ) ) + T 4 ( P ( N ) )$$

wherein Σⱼ₌₁⁵(Tj(P(1)) is the time to do all stations for the first patient,

Σᵢ₌₂^{N}(T3(P(i)) is the sum of each time the next patient has to wait until the previous patient finishes treatment to make room for the next patient (this waiting time may be significantly shorter than the prior art waiting time);

Σᵢ₌₂^{N}(T4(P(i)) is the total time for irradiating the other patients;

and T4(P(N)) is the time to dismount the final patient. The time to dismount the other patients is absorbed in the time for dealing with the other patients. The time savings over the prior art can be significant.

What is claimed is:

1. A system comprising:
carts for a plurality of patients, each of the carts dedicated and arranged to support and move each of the patients relative to the respective cart and between stations;
a mounting and immobilization station configured to allow mounting and immobilizing the patients on said carts;
an imaging station comprising an imager including image registration for registering the carts to the imager, the imager operable to produce imaging data related to the immobilized patients on said carts;
a waiting station configured to provide waiting space for said carts;
a treatment station comprising an irradiator operable to irradiate the immobilized patients on said carts according to respective treatment plans, said irradiator including at least one of image registration, optical alignment and mechanical coupling for registering the carts to the irradiator; and
a dismounting station configured to allow releasing and dismounting the patients from said carts.

2. The system according to claim 1, comprising more than one treatment station for irradiating more than one patient simultaneously.

3. A method comprising:
using the system of claim 1 to treat a plurality of patients.

4. The method according to claim 3, wherein while one of the patients is irradiated, at least one other patient is mounted, immobilized or registered to a respective cart.
